(19) United States
(12) Patent Application Publication
(10) Pub. No.: US 2005/0137626 A1
(43) Pub. Date: Jun. 23, 2005

(54) Drug delivery system and method employing external drug delivery device in conjunction with computer network

(76) Inventors: Joseph M. Pastore, Oakdale, MN (US); Jeffrey E. Stahmann, Ramsey, MN (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/742,574

(22) Filed: Dec. 19, 2003

(51) Int. Cl. A61N 1/362

(52) U.S. Cl. 607/3; 604/891.1

(57) ABSTRACT

A cardiac rhythm management system detects a condition indicative of acute decompensated heart failure and, in response, delivering a drug therapy. The system includes an implantable device communicating with a transdermal drug delivery device. The transdermal drug delivery device delivers a pharmaceutical substance in response to a detection of the condition by the implantable device. In one example, the implantable device detects the condition by monitoring a hemodynamic performance. In another example, the implantable device detects the condition by sensing a signal indicative of decompensation.

### CROSS-REFERENCE TO RELATED APPLICATION

This application is related to co-pending, commonly assigned U.S. patent application Ser. No. 10/645,823, entitled “METHOD AND APPARATUS FOR MODULATING CELLULAR METABOLISM DURING POST-ISCHEMIA OR HEART FAILURE,” filed on Aug. 21, 2003, U.S. patent application Ser. No. 10/038,936, “METHOD AND APPARATUS FOR MEASURING LEFT

VENTRICULAR PRESSURE,” filed on Jan. 4, 2002, and U.S. patent application Ser. No. 09/740,129, entitled “DRUG DELIVERY SYSTEM FOR IMPLANTABLE MEDICAL DEVICE,” filed on Dec. 18, 2000, which are hereby incorporated by reference in their entirety.

### FIELD OF THE INVENTION

This document generally relates to cardiac rhythm management systems and particularly, but not by way of limitation, to such systems employing transdermal drug delivery devices for treating heart failure.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the organs and pump it into the lungs where the blood gets oxygenated. The body's metabolic need for oxygen increases with the body's physical activity level. The pumping functions are accomplished by contractions of the myocardium (heart muscles). An increase in the body's metabolic need for oxygen is satisfied primarily by a higher frequency of the contractions, i.e., a higher heart rate. In a normal heart, the sinoatrial node, the heart's natural pacemaker, generates electrical impulses, known as action potentials, that propagate through an electrical conduction system to various regions of the heart to excite myocardial tissues in these regions. Coordinated delays in the propagations of the action potentials in a normal electrical conduction system cause the various regions of the heart to contract in synchrony such that the pumping functions are performed efficiently.

A blocked or otherwise damaged electrical conduction system causes irregular contractions of the myocardium, a condition generally known as arrhythmia. Arrhythmia reduces the heart's pumping efficiency and hence, diminishes the blood flow to the body. A deteriorated myocardium has decreased contractility, also resulting in diminished blood flow. A heart failure patient usually suffers from both a damaged electrical conduction system and a deteriorated myocardium. The diminished blood flow results in insufficient blood supply to various body organs, preventing these organs to function properly and causing various symptoms. For example, in a patient suffering acute decompensated heart failure, an insufficient blood supply to the kidneys results in fluid retention and edema in the lungs and peripheral parts of the body, a condition referred to as decompensation.

The patient suffering acute decompensated heart failure can benefit from cardiac pacing and/or drug therapy. Cardiac pacing restores the function of the electrical conduction system to a certain degree. Certain medications, such as cardiotonic drugs and diuretics, are known to strengthen the cardiac muscles and reduce the fluid retention, thereby stopping or slowing the decompensation process. Because acute decompensated heart failure progresses rapidly after onset, a fast response upon early indications is required.

For these and other reasons, there is a need for an efficient method and system to detect decompensation events deliver pacing and drug therapies to compensating a heart failure patient.

### SUMMARY

A cardiac rhythm management system detects a condition indicative of acute decompensated heart failure and, in response, delivering a drug therapy. The system includes an implantable device communicating with a transdermal drug delivery device. The transdermal drug delivery device delivers a pharmaceutical substance in response to a detection of the condition by the implantable device. In one example, the implantable device detects the condition by monitoring a hemodynamic performance. In another example, the implantable device detects the condition by sensing a signal indicative of decompensation.

In one embodiment, a system includes a transdermal drug delivery device and an implantable cardiac rhythm management (CRM) device. The implantable CRM device communicates with the transdermal drug delivery device and includes a heart failure detector and a drug delivery controller. The heart failure detector detects an acute decompensated heart failure and produces an alert signal in response to a detection of the acute decompensated heart failure. The drug delivery controller receives an external user command and controls the transdermal drug delivery device based on at least the alert signal and the external user command.

In one embodiment, a method provides for detection of acute decompensated heart failure and a response to the detection, by using an implantable CRM device and a transdermal drug delivery device. The acute decompensated heart failure is detected using the implantable CRM device. An external user command controlling a drug delivery, transmitted to the implantable CRM device from an external device, is also detected. A drug control signal is produced based on at least the detected acute decompensated heart failure and the external user command, and transmitted to the transdermal drug delivery device. In response, a drug is delivered from the transdermal drug delivery device.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their equivalents.

### BRIEF DESCRIPTION OF THE DRAWINGS

In the drawings, which are not necessarily drawn to scale, like numerals describe similar components throughout the several views. The drawings illustrate generally, by way of example, but not by way of limitation, various embodiments discussed in the present document. The drawing are for illustrative purposes only and not to scale nor anatomically accurate.

FIG. 1 is an illustration of an embodiment of a transdermal drug delivery system and portions of an environment in which it is used.

FIG. 2A is a block diagram showing one embodiment of the circuit of portions of the transdermal drug delivery system such as shown in FIG. 1.

FIG. 2B is a block diagram showing one embodiment including additional details of the circuit of FIG. 2A.

FIG. 3 is a flow chart illustrating an embodiment of a method for delivering a drug to treat acute decompensated heart failure using the transdermal drug delivery system such as shown in FIG. 1.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their equivalents.

It should be noted that references to “an”, “one”, or “various” embodiments in this disclosure are not necessarily to the same embodiment, and such references contemplate more than one embodiment.

This document discusses a cardiac rhythm management (CRM) system that includes a transdermal drug delivery device to treat acute decompensated heart failure. In one embodiment, pharmaceutical agents are transdermally delivered in conjunction with electrical therapy. The CRM system detects certain physiological signals and/or events indicative of acute heart failure decompensation and, in response, delivers one or more pharmaceutical agents to strengthen myocardial tissues and/or reduce fluid retention in the body. The term “pharmaceutical agents,” as used in this document, include agents that are chemical, biochemical, and/or biologic in nature.

Pharmaceutical agents within the scope of the present subject matter include all chemical, biochemical, and biological agents used to treat heart failure including all treatable symptoms of or related to heart failure. Examples of such agents include anti-hypertensive agents, anti-arrhythmic agents, pressors, vasopressors, vasodilators, anti-hyperlipidemic agents, anti-anginal agents, inotropic agents, diuretics, volume expanders, thrombolytics, anti-platelet agents, beta-blockers, angiotensin converting enzyme (ACE) inhibitors, and angiotensin receptor blockers, or any combination thereof, including but not limited to diuretics such as thiazides, e.g., hydrochlorothizide, loop duretics, e.g., furosemide, and potassium-sparing agents, e.g., amiloride, sprionolactone and triamterene and hydrochlorothiazide, beta-blockers such as bisoprolol, carvedilol, labetolol and metoprolol, angiotensin-converting enzyme inhibitors such as benazepril, captopril, enalapril, fosinopril, lisinopril, perindopril, quinapril, ramipril, trandolapril, delapril, pentopril, moexipril, spirapril, temocapril, and imidapril, calcium channel blockers, alpha blockers, angiotensin II antagonists, e.g., losartan, statins, e.g., atorvastatin, pitavastatin, and pravastatin, or other lipid lowering agents, moxonidine, dihydropyridines, e.g., amlodipine, class III and IV antiarrhythmics, e.g., amiodarone, azimilide, sotalol, dofetilide, and ubutilide, aspirin, selective non-adrenergic imidazoline receptor inhibitors, hebivolol, vasopeptidase inhibitors, e.g., fasidotritat, omapatrilat, samapatrilat, substrates, inhibitors or inducers of cytochrome P450 enzymes, lidocaine, warfarin, oligonucleotides (sense or antisense), natriuretic peptides such as ANP, BNP, NT pro BNP, CNP, and DNP, colforsin daropate hydrochloride (forskilin derivative), antagonists of platelet integrin IIb/IIIa receptors, e.g., abciximab and trofiblant, reteplase, P2 receptor antagonists, e.g., ticlopidine and clopidrogel, mibefradil, hirudin, acetylcholinesterase inhibitors, cardiac glycosides, e.g., digoxin and digitoxin, bradykinin, neutral endopeptidease inhibitors, e.g., neprilysin, direct-acting vasodilators, e.g., hydralazine, nitroglycerin, sodium nitroprusside, catecholamines, dobutramine and dopamine, phosphodiesterase inhibitors, e.g., amrinone and milrinone, TNFa, pentoxifylline, growth hormone, cytokine inhibitors, aldosterone receptor antagonists, calcium sensitizers, nesiritide, 3,5-dicodothyropropionic acid, etomoxir, endothelin receptor antagonists, chlorthiadone, doxazosin, cilostazol, rilmenidine, ticlopidine, dihydropines such as nifedipine and nisoldipine, timolol, propanolol, verapamil, diltiazem, lisinopril, noopept (N-phenylacetyl-L-polyglycine ethylester), cariporide, geldanamycin, radicicol, ibudilast, selective delta (1) agonists such as 2-methyl-4a-alpha-(3-hydroxy-phenyl)-1,2,3,4,4a,5,12,12a-alpha-octahydroquinolinol [2,3,3-g]isoquinoline, monophosphoryl lipid A, RC552, adenosine, adenosine receptor agonists, adenosine triphosphate sensitive channel openers, dipyridamole, fibroblast growth factor, atenolol, ezetimibe, lerosimendan, sirolimus, paclitaxil, actinomycin D, dexamethasone, tacrolimeus, everolimus, estradiol, quinapril, tranilast, antiopeptin, trapidil, lacidipine, thiazolidinediones, fenofibrate, lacidipine, nebrivolol, nicotinic acid, probucal, rosuvastatin, gemfibrozil, glitazones, indobugen, alpha-tocopherol, dypiridamole, resins, e.g., cholestyramine and colestipol, bezafibrate, or listat, niacin, heparin, e.g., low molecular weight heparins such as dalteparin sodium and nadroparin calcium, bivalirucin, nitroglycerin, nicorandil, denopamine, eptifibatide, xemilofiban, or bofiban, trimetazidine, nicorandil, dalteparin, and isosorbide 5-mononitrate. Additional pharmaceutical agents may be considered based on evidence of their direct or indirect roles in preventing or reducing injury or hemodynamic compromise related to myocardial infarction and/or heart failure. Examples of such pharmaceutical agents include, but are not limited to, L-arginine; nitric oxide (NO); NO derivatives such as nitroxl anion (HNONO—) and peroxynitrite (ONOO—); iNOS, eNOS, and inhibitors such as nitro-L-arginine methyl ester; NO donors such as diethylamine (DEA) NO and nitroglycerin (NTG); and interleukins and interleukin inhibitors.

The CRM system discussed in this document includes a transdermal drug delivery device to deliver a drug including one or more pharmaceutical agents in response to a detected acute decompensated heart failure. Specific examples of the one or more pharmaceutical agents include, but are not limited to, all pharmaceutical agents discussed in this document.

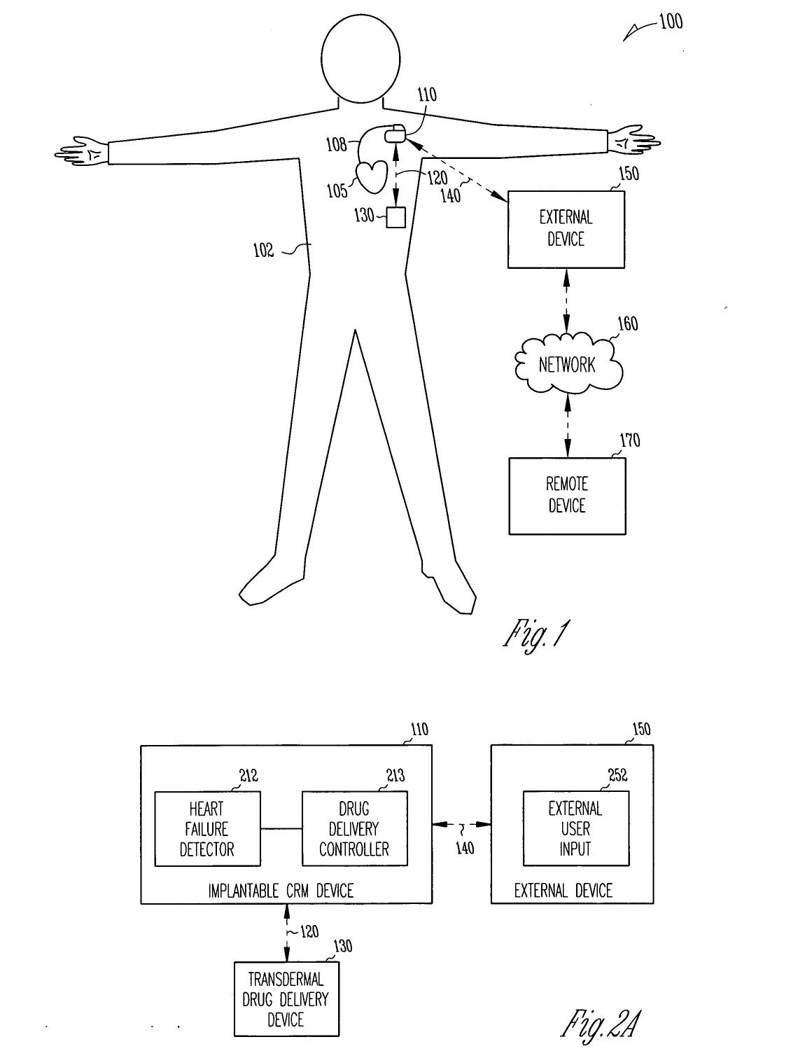

FIG. 1 is an illustration of an embodiment of a transdermal drug delivery system 100 and portions of an environment in which it is used. System 100 includes, among other things, an implantable CRM device 110, a lead system 108, a transdermal drug delivery device 130, an external device 150, a network 160, and a remote device 170. As shown in FIG. 1, implantable CRM device is implanted in a body 102. Transdermal drug delivery device 130 is attached to the skin surface of body 102 at a site near heart 105. Lead system 108 includes one or more leads providing for electrical connections between heart 105 and implantable CRM device 110. A communication link 120 allows signal transmission between implantable CRM device 110 and transdermal drug delivery device 130. A telemetry link 140 provides for bidirectional communication between implantable CRM device 110 and external device 150. Network 160 provides for bidirectional communication between external device 150 and remote device 170.

System 100 allows a drug delivery to be triggered by any one of implantable CRM device 110, external device 150, and remote device 170. In one embodiment, implantable CRM device 110 triggers a drug delivery upon detecting a predetermined signal or condition. External device 150 triggers a drug delivery upon receiving an external user command from the patient wearing implantable CRM device 110 and transdermal drug delivery device 130 or from another person such as a relative, a friend, or a physician/caregiver. The patient enters the external user command when he or she detects an acute abnormal condition indicative of heart failure. Another person caring for the patient may also enter the external user command upon a request by the patient or an observation of symptoms of acute decompensated heart failure. Remote device 170 triggers a drug delivery upon receiving a remote user command from a physician/caregiver, who has been given information about the patient's condition and symptoms. In other embodiments, external device 150 and/or remote device 170 process signals and/or a condition detected by implantable CRM device 110 to determine whether to trigger a drug delivery. Thus, system 100 is used for an acute treatment for relief of heart failure decompensation as soon as heart failure is detected by any one of implantable CRM device 1 10, the patient, or the physician/caregiver.

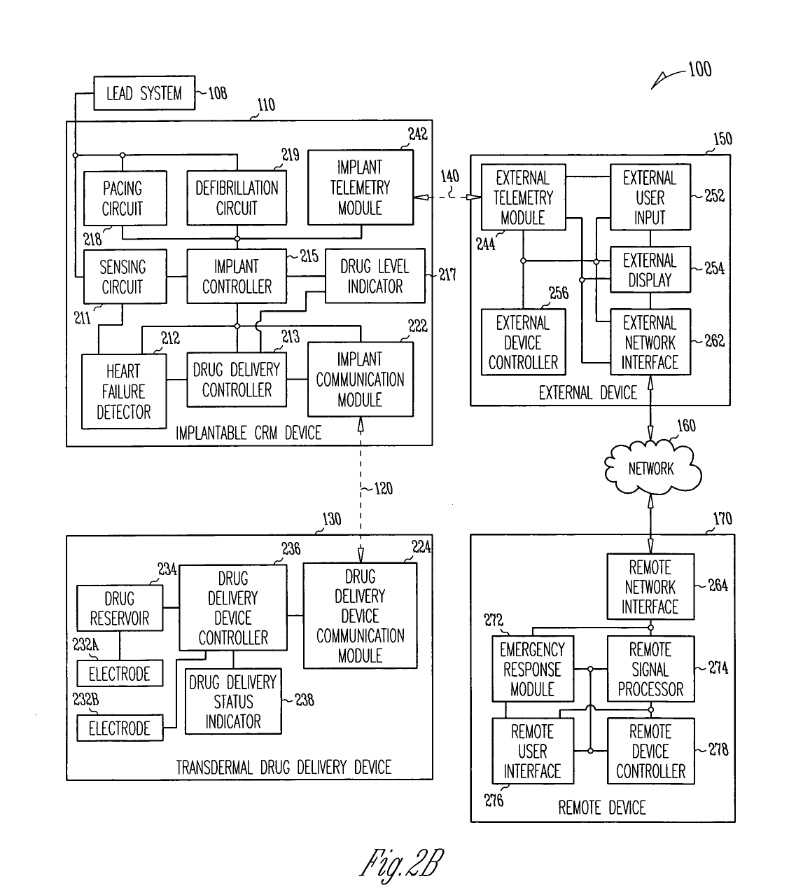

FIG. 2A is a block diagram showing one embodiment of the circuit of portions of system 100 including implantable CRM device 1 10, lead system 108, transdermal drug delivery device 130 and external device 150. Implantable CRM device 110 communicates with transdermal drug delivery device 130 via telemetry link 120. External device 150 communicates with implantable CRM device via telemetry link 140.

Implantable CRM device includes a heart failure detector 212 and a drug delivery controller 213. Heart failure detector 212 detects a condition indicative of acute decompensated heart failure. In response to a detection of the condition, heart failure detector 212 produces an alert signal indicating the detection. In one embodiment, the alert signal includes information indicative of a status or degree of the heart failure. Heart failure results in diminished blood flow from the heart as measured by cardiac output or stroke volume. Cardiac output is the amount of blood pumped by the heart during a unit period of time. Stroke volume is the amount of blood pumped during each contraction or stroke. Decompensated heart failure occurs when the heart becomes significantly weakened such that the body's compensation mechanism cannot restore a normal cardiac output. One principal consequence of the decompensated heart failure is that the heart fails to provide the kidneys with sufficient blood to support normal renal function. As a result, a patient suffering decompensated heart failure progressively develops pulmonary and peripheral edema, a process referred to as decompensation. Thus, parameters indicative of hemodynamic performance as well as parameters indicative of decompensation indicate acute decompensated heart failure.

In one embodiment, heart failure detector 212 includes an implantable impedance sensor to measure pulmonary impedance, or impedance of a portion of the thoracic cavity. Heart failure detector 212 produces the alert signal when the impedance is out of its normal range. For example, pulmonary edema, i.e., fluid retention in the lungs resulting from the decreased cardiac output, increases the pulmonary or thoracic impedance. In one specific embodiment, heart failure detector 212 produces the alert signal when the pulmonary or thoracic impedance exceeds a predetermined threshold impedance. In one embodiment, the impedance sensor is a respiratory sensor that senses the patient's minute ventilation. An example of an impedance sensor sensing minute ventilation is discussed in U.S. Pat. No. 6,459,929, “IMPLANTABLE CARDIAC RHYTHM MANAGEMENT DEVICE FOR ASSESSING STATUS OF CHF PATIENTS,” assigned to Cardiac Pacemakers, Inc., which is incorporated herein by reference in its entirety.

In one embodiment, heart failure detector 212 includes a pressure sensor. Acute decompensated heart causes pressures in various portions of the cardiovascular system to deviate from their normal ranges. Heart failure detector 212 produces the alert signal when a pressure is outside of its normal range. Examples of the pressure sensor include a left atrial (LA) pressure sensor, a left ventricular (LV) pressure sensor, an artery pressure sensor, and a pulmonary artery pressure sensor. Pulmonary edema results in elevated LA and pulmonary arterial pressures. A deteriorated LV results in decreased LV and arterial pressures. In various embodiments, heart failure detector 212 produces the alert signal when the LA pressure exceeds a predetermined threshold LA pressure level, when the pulmonary arterial pressure exceeds a predetermined threshold pulmonary arterial pressure level, when the LV pressure falls below a predetermined threshold LV pressure level, and/or when the arterial pressure falls below a predetermined threshold LV pressure level. In other embodiments, heart failure detector 212 derives a parameter from one of these pressures, such as a rate of change of a pressure, and produces the alert signal when the parameter deviates from its normal range. In one embodiment, the LV pressure sensor senses the LV pressure indirectly, by sensing a signal having known or predictable relationships with the LV pressure during all or a portion of the cardiac cycle. Examples of such a signal include an LA pressure and a coronary vein pressure. One specific example of measuring the LV pressure using a coronary vein pressure sensor is discussed in U.S. patent application Ser. No. 10/038,936, “METHOD AND APPARATUS FOR MEASURING LEFT VENTRICULAR PRESSURE,” filed on Jan. 4, 2002, assigned to Cardiac Pacemakers, Inc., which is hereby incorporated by reference in its entirety.

In one embodiment, heart failure detector 212 includes a cardiac output or stroke volume sensor. Examples of stroke volume sensing are discussed in U.S. Pat. No. 4,686,987, “BIOMEDICAL METHOD AND APPARATUS FOR CONTROLLING THE ADMINISTRATION OF THERAPY TO A PATIENT IN RESPONSE TO CHANGES IN PHYSIOLOGIC DEMAND,” and U.S. Pat. No. 5,284,136, “DUAL INDIFFERENT ELECTRODE PACEMAKER,” both assigned to Cardiac Pacemakers, Inc., which are incorporated herein by reference in their entirety. Heart failure detector 212 produces the alert signal when the stroke volume falls below a predetermined threshold level.

In one embodiment, heart failure detector 212 includes a neural activity sensor to detect activities of the sympathetic nerve and/or the parasympathetic nerve. A significant decrease in cardiac output immediately stimulates sympathetic activities, as the autonomic nervous system attempts to compensate for deteriorated cardiac function. Sympathetic activities sustain even when the compensation fails to restore the normal cardiac output. In one specific embodiment, the neural activity sensor includes a neurohormone sensor to sense a hormone level of the sympathetic nerve and/or the parasympathetic nerve. Heart failure detector 212 produces the alert signal when the hormone level exceeds a predetermined threshold level. In another specific embodiment, the neural activity sensor includes an action potential recorder to sense the electrical activities in the sympathetic nerve and/or the parasympathetic nerve. Heart failure detector 212 produces the alert signal when the frequency of the electrical activities in the sympathetic nerve exceeds a predetermined threshold level. Examples of direct and indirect neural activity sensing are discussed in U.S. Pat. No. 5,042,497, “ARRHYTHMIA PREDICTION AND PREVENTION FOR IMPLANTED DEVICES,” assigned to Cardiac Pacemakers, Inc., which is hereby incorporated by reference in its entirety.

In one embodiment, heart failure detector 212 includes a heart rate variability detector. Patients suffering acute decompensated heart failure exhibit abnormally low heart rate variability. An example of detecting the heart rate variability is discussed in U.S. Pat. No. 5,603,331, “DATA LOGGING SYSTEM FOR IMPLANTABLE CARDIAC DEVICE,” assigned to Cardiac Pacemakers, Inc., which is incorporated herein by reference in their entirety. Heart failure detector 212 produces the alert signal when the heart rate variability falls below a predetermined threshold level.

In one embodiment, heart failure detector 212 includes a renal function sensor. Acute decompensated heart failure results in peripheral edema primarily because of fluid retention of the kidneys that follows the reduction in cardiac output. The fluid retention is associated with reduced renal output, decreased glomerular filtration, and formation of angiotensin. Thus, in one specific embodiment, the renal function sensor includes a renal output sensor to sense a signal indicative of the renal output. Heart failure detector 212 produces the alert signal when the sensed renal output falls below a predetermined threshold. In another specific embodiment, the renal function sensor includes a filtration rate sensor to sense a signal indicative of the glomerular filtration rate. Heart failure detector 212 produces the alert signal when the sensed glomerular filtration rate falls below a predetermined threshold. In yet another specific embodiment, the renal function sensor includes a chemical sensor to sense a signal indicative of angiotensin II levels. Heart failure detector 212 produces the alert signal when the sensed angiotensin II levels exceed a predetermined threshold level.

In one embodiment, heart failure detector 212 includes an acoustic sensor being a heart sound sensor and/or a respiratory sound sensor. Acute decompensated heart failure causes abnormal cardiac and pulmonary activity patterns and hence, deviation of heart sounds and respiratory sounds from their normal ranges of pattern and/or amplitude. Heart failure detector 212 produces the alert signal when the heart sound or respiratory sound is out of its normal range. For example, detection of the third heard sound (S3) is known to indicate heart failure. In one specific embodiment, heart failure detector 212 produces the alert signal when the S3 amplitude exceeds a predetermined threshold level.

Embodiments of heart failure detector 212 are discussed in this document by way of example, but not by way of limitation. Other methods and sensors for directly or indirectly detecting the acute decompensated heart failure, as known to those skilled in the art, are useable as heart failure detector 212.

Implantable CRM device 110 includes a hermetically sealed metal can to house at least portion of the electronics of the device. In one embodiment, heart failure detector 212 resides within the metal can. In another embodiment, heart failure detector 212 is outside of the metal can.

External device 150 includes an external user input 252 to receive an external user command for delivering the drug. The user command is transmitted to implantable CRM 140 device, via telemetry link 140, to be received by drug delivery controller 213. Upon receiving at least one of the alert signal from heart failure detector 212 and the external user command from external device 150, drug delivery controller 213 generates a drug control signal. The drug control signal is transmitted to transdermal drug delivery device 130, via telemetry link 120, to trigger a drug delivery.

FIG. 2B is a block diagram showing one embodiment including additional details of the circuit of FIG. 2A. Implantable CRM device 110 as shown in FIG. 2B includes pacing and defibrillation capabilities. In addition to drug delivery, examples of therapies delivered by implantable CRM device 110 include, but are not limited to, bradyarrhythmia pacing, anti-tachyarrhythmia pacing, atrial and/or ventricular cardioversion/defibrillation, cardiac resynchronization therapy, and cardiac remodeling control. However, the pacing and defibrillation capabilities are not necessary for system 100 to perform drug delivery, and hence, are not necessary elements of implantable CRM device 110. In other words, implantable CRM device 110 can be an implantable pacemaker and/or defibrillator with additional functions including control of drug delivery, or it can be a dedicated implantable drug delivery processor or controller.

In one embodiment, implantable CRM device 110 includes a sensing circuit 211, a heart failure detector 212, a drug delivery controller 213, a drug level indicator 217, a pacing circuit 218, a defibrillation circuit 219, an implant controller 215, an implant communication module 222, and an implant telemetry module 242.

Sensing circuit 211 senses one or more intracardiac electrogram through a lead of lead system 108. In one embodiment, sensing circuit 211 senses both atrial and ventricular electrograms. In another embodiment, sensing circuit 211 senses multiple ventricular electrograms. Pacing circuit 218 delivers pacing pulses to one or more cardiac regions as controlled by implant controller 215. Defibrillation circuit 219 delivers cardioversion or defibrillation shocks to one or more cardiac regions as controlled by implant controller 215. Heart failure detector 212 detects a condition indicative of acute decompensated heart failure and produces an alert signal indicating each detection, as discussed above with reference to FIG. 2A.

Drug delivery controller 213 includes a command receiver to receive the external user command transmitted from external device 150. Upon receiving at least one of the alert signal from heart failure detector 212, an external user command from external device 150, and a remote user command from remote device 170, drug delivery controller 213 generates a drug control signal. The drug control signal is transmitted through communication link 120 to transdermal drug delivery device 130 to trigger a drug delivery. After the drug delivery, drug level indicator 217 measures or estimates a blood drug concentration of the drug delivered to produce an indication of the blood drug concentration. In one embodiment, drug level indicator 217 includes a drug level detector that measures the blood drug concentration. In another embodiment, drug level indicator 217 includes a sensor measuring a physiological parameter indicative of the blood drug concentration. If drug level indicator 217 produces an indication of a blood drug concentration that is below a predetermined minimum level, drug delivery controller 213 produces a further drug control signal to continue the drug delivery or start another drug delivery. Implant controller 215 provides for overall control and signal processing for implantable CRM device 110. Implant communication module 222 provides for a signal transmission interface allowing implantable CRM device 110 to communicate with transdermal drug delivery device 130, such as to transmit the drug control signal, via communication link 120. Implant telemetry module 242 provides for a telemetry interface allowing implantable CRM device 110 to communicate with external device 150 via telemetry link 140.

Lead system 108 includes one or more pacing leads, defibrillation leads, pacing-defibrillation leads, or any combination of such leads. It allows sensing of electrical signals from heart 105 and/or delivery of pacing pulses and/or defibrillation shocks to heart 105. In one embodiment, lead system 108 includes one or more transvenous leads each having at least one sensing-pacing electrode disposed within heart 105. In one embodiment, lead system 108 includes one or more epicardial leads each having at least one sensing-pacing electrode disposed on heart 105. On one embodiment, lead system 108 includes one or more leads each having at least one sensor such as an accelerometer or a metabolic sensor. In one specific embodiment, lead system 108 includes one or more leads each having a metabolic sensor disposed in a blood pool when the lead is implanted.

Transdermal drug delivery device 130 includes electrodes 232A-B, drug reservoir 234, drug delivery device controller 236, drug delivery status indicator 238, and drug delivery communication module 224. One specific example of transdermal drug delivery device 130 is discussed in U.S. Pat. No. 6,361,522, entitled “DRUG DELIVERY SYSTEM FOR IMPLANTABLE CARDIAC DEVICE,” assigned to Cardiac Pacemakers, Inc., which is incorporated herein by reference in its entirety. In one embodiment, transdermal drug delivery device 130 is a skin patch allowing electrically controlled transdermal drug delivery by, for example, iontophoresis, electroporation, electrorepulsion, or electro-osmosis. The skin patch is to be attached on a surface site of body 102 near heart 105. Electrodes 232A and 232B are skin-contact electrodes. Drug reservoir 234 contains the drug, which includes one or more pharmaceutical agents treating acute decompensated heart failure. Drug delivery status indicator 238 allows the patient and any other person such as a physician/caregiver to monitor, for example, whether the drug is being delivered and/or the amount of the drug remains in drug reservoir 234. Drug delivery device controller 236 controls the overall operation of transdermal drug delivery device 130. In one embodiment, drug delivery device controller 236 generates an electrical potential to cause the drug delivery upon receiving and/or decoding the drug control signal. Drug delivery communication module 224 provides for a signal transmission interface allowing transdermal drug delivery device 130 to communicate with implantable CRM device 110, such as to receive the drug control signal, via communication link 120. In one embodiment, transdermal drug delivery device 130 includes a reservoir drug level detector to detect the level of drug remaining in drug reservoir 234, and produce a warning signal if the reservoir drug level reaches a minimum level. The warning signal is transmitted through implantable CRM device 110 to external device 150 to inform the user of a need to replenish drug reservoir 234.

Communication link 120 is supported by implant telemetry module 222 and drug delivery communication module 224. It allows communications between implantable CRM device 110 and transdermal drug delivery device 130. In one embodiment, communication link 120 is a telemetry link. In another embodiment, implantable CRM device 110 transmits electrical signals representative of the drug control signal into tissue of body 102, to be sensed through electrodes 232A-B and hence received by transdermal drug delivery device 130. In this embodiment, communication link 120 uses body 102 as the conductive medium for conducting electrical signals. One specific example of such a communication link is discussed in U.S. patent application Ser. No. 09/740,129, entitled “DRUG DELIVERY SYSTEM FOR IMPLANTABLE MEDICAL DEVICE,” filed on Dec. 18, 2000, assigned to Cardiac Pacemakers, Inc., which is incorporated herein by reference in its entirety.

External device 150 includes an external user input 252, an external display 254, an external device controller 256, an external telemetry module 244, and an external network interface 262. External user input 252 receives the external user command from the patient or another person. In a further embodiment, it also receives other commands or instructions to control the operation of transdermal drug delivery device 130 and/or implantable CRM device 110. External device 150 transmits the external user command to implantable CRM device 110, resulting in a production of the drug control signal by drug delivery controller 213. In one embodiment, external device 150 also transmits the external user commands to remote device 170. In response, a remote user command directing a drug delivery may return from remote device 170. External device 150 relays the remote user command to implantable CRM device 110, resulting in a production of the drug control signal by drug delivery controller 213. External user input 252 includes a switch. In one embodiment, external user input 252 includes a push button. The patient pushes it, for example, when feeling an onset of acute decompensated heart failure. In another embodiment, external user input 252 includes a voice controlled switch such that the patient may orally order a drug delivery. External telemetry module 244 provides for a telemetry interface allowing external device 150 to communicate with implantable CRM device 110 via telemetry link 140. External network interface 262 provides for a network interface allowing external device 150 to communicate with remote device 170 via network 160.

Telemetry link 140 is a wireless bidirectional data transmission link supported by implant telemetry module 242 and external telemetry module 244. In one embodiment, telemetry link 140 is an inductive couple formed when two coils—one connected to implant telemetry module 242 and the other connected to external telemetry module 244—are placed near each other. In this embodiment, the patient or another person places external device 150 on body 102 over implantable CRM device 110. In another embodiment, telemetry link 140 is a far-field radio-frequency telemetry link allowing implantable CRM device 110 and external device 252 to communicate over a telemetry range that is at least ten feet.

Remote device 170 includes an emergency response module 272, a remote signal processor 274, a remote user interface 276, a remote device controller 278, and a remote network interface 264. By executing one or more predetermined algorithms, remote signal processor 274 processes signals transmitted from external device 150 and signals transmitted from implantable CRM device 110. Emergency response module 272 contacts an emergency response unit, such as by calling 911 (in the United States), in response to an emergency situation as determined by one of implantable CRM device 110, external device 150, and remote device 170. In one embodiment, external device 150 transmits the external user command to remote device 170 as a request for contacting the emergency response unit through emergency response module 272. In another embodiment, remote signal processor 274 analyzes signals acquired by implantable CRM device 110 and transmitted to remote device 170, such as a portion of the electrogram sensed by sensing circuit 211, to determine the need for contacting the emergency response unit. In yet another embodiment, a physician/caregiver observes signals and/or the result of the analysis through remote user interface 276 to determine whether to contact the emergency response unit. Remote user interface 276 allows the physician/caregiver to enter a remote user command to be transmitted to transdermal drug delivery device 130. It also allows physician/caregiver to enter the remote user command to be transmitted to implantable CRM device 110 for a delivery or adjustment of pacing and/or defibrillation therapy. Remote device controller 278 controls the overall operation of remote device 170. In one embodiment, remote device controller 278 generates commands controlling one or more of transdermal drug delivery device 130, implantable CRM devicel 10, and external device 150 based on the received signals such as the portion of electrogram and the external user command. In one embodiment, remote device controller 278 executes an automatic algorithm to determine whether to issue a drug delivery command or to issue an electrical therapy (pacing and/or defibrillation, including cardiac resynchronization and/or remodeling control) command, such as when a physician/caregiver is not immediately available. Remote network interface 264 provides for an interface allowing communication between remote device 170 and external device 150 via network 160.

Network 160 provides long distance bidirectional communication between external device 150 and remote device 170. It allows management of multiple implantable devices, such as implantable CRM device 110 and transdermal drug delivery device 130, from a central facility at a remote location. In one embodiment, this allows prompt response by a physician/caregiver at the central facility as demanded by the condition of a patient. In one embodiment, network 160 is based on a wireless communications system. In another embodiment, network 160 is based on a wired communications system. In one embodiment, network 160 utilizes portions of a standard communications system such as the Internet, a telephone system, or a radio frequency telemetry system.

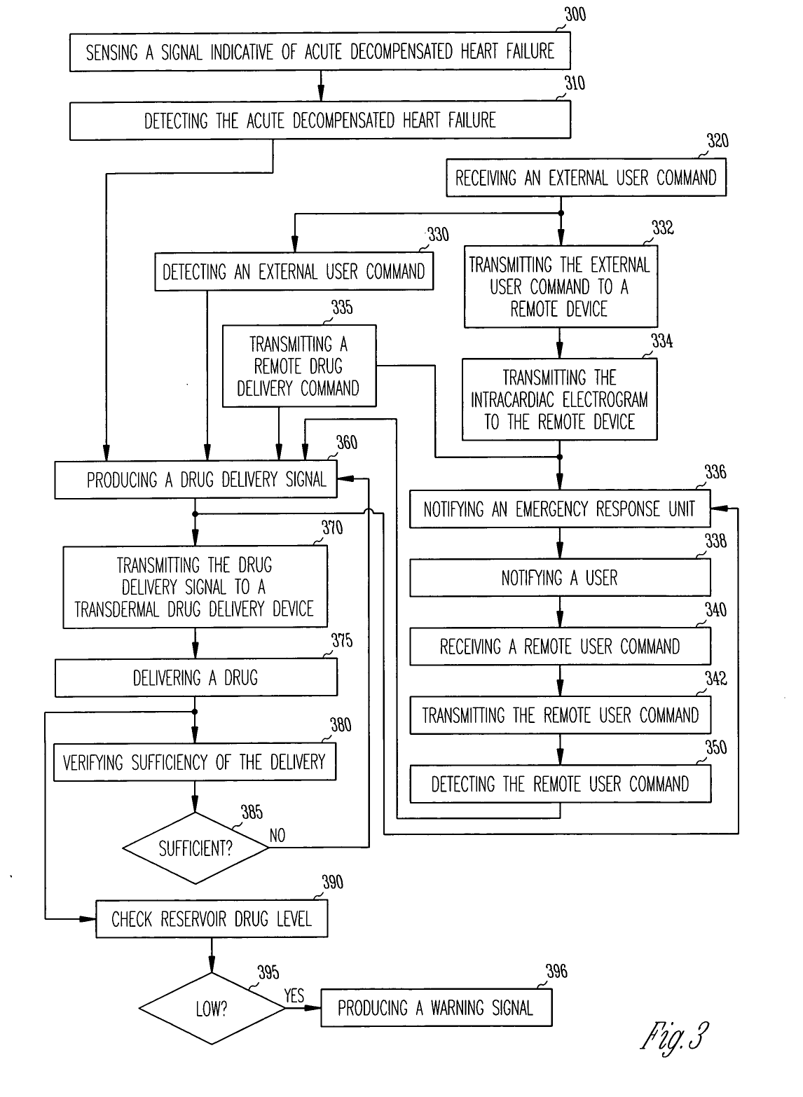

FIG. 3 is a flow chart illustrating an embodiment of a method for delivering a drug using system 100. Heart failure detector 212 senses a signal indicative of acute decompensated heart failure at 300. At 310, the acute decompensated heart failure is detected. In response, heart failure detector 212 produces a heart failure indicating signal and sends it to drug delivery controller 213.

External user input 252 receives an external user command at 320. The patient enters the external user command when he or she feels a need for an immediate treatment. Alternatively, another person, such as a physician/caregiver, an attendant, or a relative, enter the external user command after acquiring information about the patient's symptoms and determining that the patient should receive an immediate drug therapy. For example, the physician/caregiver enters the external user command in response to an observation of symptoms of decompensation. After external device 150 transmits the external user command to implantable CRM device 110, drug delivery controller 213 detects the external user command at 330.

In one embodiment, in addition to transmitting the external user command to implantable CRM device 110, external device 150 transmits the external user command to remote device 170 though network 160 at 332. In one embodiment, remote device 170 also receives signals acquired by implantable CRM device 110, such as the electrogram, and transmits the signals to remote device 170 at 334. In one embodiment, after receiving the external user command and/or analyzing the signals acquired by implantable CRM device 110, remote device 170 notifies an emergence response unit, such as by calling 911 (as in the United States), at 336. In one embodiment, after receiving the external user command and/or analyzing the signals acquired by implantable CRM device 110, remote device 170 automatically produces a remote drug delivery command that is transmitted to implantable CRM device 110 through external device 150 at 335. In one embodiment, after receiving the external user command and/or analyzing the signals acquired by implantable CRM device 110, remote device 170 also notifies a user such as a physician/caregiver at 338. After the user makes a decision, remote device 170 receives a remote user command at 340. The remote user command directs a drug delivery and/or a delivery or adjustment of pacing (including such as cardiac resynchronization and remodeling control) therapy. In one embodiment, a physician/caregiver near remote device 170 enters the remote user command based on the information he acquired regarding the patient's symptoms. Remote device 170 transmits the remote to external device 150 through network 160, and external device 150 relays the remote user command to implantable CRM device 110 at 342. Drug delivery controller 213 of implantable CRM device 110 detects the remote user command at 350.

Drug delivery controller 213 produces a drug control signal at 360, upon the detection of at least one of the acute decompensated heart failure, the external user command, the remote drug delivery command, and the remote user command. In one embodiment, the drug control signal is also transmitted to remote device 170 for notifying the emergency response unit and/or the user. Implantable CRM device 110 transmits the drug control signal to transdermal drug delivery device 130 at 370. In one embodiment, implantable CRM device 110 transmits the drug control signal the drug control signal via a telemetry link between implantable CRM device 110 and transdermal drug delivery device 130. In another embodiment, implantable CRM device 110 transmits an electrical signal representing the drug control signal to transdermal drug delivery device 130 via tissue conduction. In one specific embodiment, implantable CRM device 110 transmits a voltage signal representing the drug control signal into tissue of body 102 to be detected by transdermal drug delivery device 130.

In response to the drug control signal, transdermal drug delivery device 130 delivers a drug into tissue at 375. In one embodiment, drug level indicator 217 verifies that a sufficient amount of the drug has been delivered at 380, by detecting a signal indicative of a blood drug concentration. In one embodiment, this includes measuring a blood drug concentration directly. In another embodiment, this includes sensing a signal indicative of the body's immediate biological response to the drug therapy. If drug level indicator 217 indicates that the blood drug concentration is below a predetermined level at 385, it produces an insufficiency alert signal and transmits it to drug delivery controller 213. Upon detection of the insufficiency alert signal, drug delivery controller 213 produces an additional drug control signal, and steps 360, 370, 375, 380, and 385 are repeated until the drug level indicator 217 indicates that the blood drug concentration reaches the predetermined level at 385, or until a predetermined maximum dosage is reached. In one embodiment, after each drug delivery at 375, transdermal drug delivery device 130 checks the remaining drug level in drug reservoir 234 at 390. If the drug level is low, such as below a specified minimum level, at 395, transdermal drug delivery device 130 produces a warning signal at 396 and sends the warning signal through implantable CRM device 110 to external device 150 and/or remote device 170 to inform the user of a need to replenish drug reservoir 234.

It is to be understood that the above detailed description is intended to be illustrative, and not restrictive. Although the present therapy is described in the example of cardiac therapy, it is understood that many other applications are possible. Systems 100 may be generally applied in drug delivery controlled by a condition detected or a signal sensed from a person. Other embodiments, including any possible permutation of the system components discussed in this document, will be apparent to those of skill in the art upon reading and understanding the above description. The scope of the invention should, therefore, be determined with reference to the appended claims, along with the full scope of equivalents to which such claims are entitled.

1. A system, comprising:
a transdermal drug delivery device; and
an implantable cardiac rhythm management (CRM) device communicatively coupled to the transdermal drug delivery device, the implantable CRM device including:
a heart failure detector to detect an acute decompensated heart failure and produce an alert signal in response to a detection of the acute decompensated heart failure; and
a drug delivery controller coupled to the heart failure detector, the drug delivery controller including a command receiver to receive an external user command and adapted to control the transdermal drug delivery device based on at least the alert signal and the external user command.

2. The system of claim 1, wherein the heart failure detector comprises an impedance sensor adapted to measure pulmonary impedance.

3. The system of claim 1, wherein the heart failure detector comprises a respiratory sensor to measure a signal indicative of minute ventilation.

4. The system of claim 1, wherein the heart failure detector comprises an impedance sensor adapted to measure impedance of a thoracic cavity.

5. The system of claim 1, wherein the heart failure detector comprises a pressure sensor.

6. The system of claim 5, wherein the heart failure detector comprises a left atrial pressure sensor.

7. The system of claim 5, wherein the heart failure detector comprises a left ventricular pressure sensor.

8. The system of claim 5, wherein the heart failure detector comprises an artery pressure sensor.

9. The system of claim 5, wherein the heart failure detector comprises a pulmonary artery pressure sensor.

10. The system of claim 1, wherein the heart failure detector comprises a stroke volume sensor.

11. The system of claim 1, wherein the heart failure detector comprises a neural activity sensor adapted to detect activities of a sympathetic nerve.

12. The system of claim 11, wherein the neural activity sensor comprises a neurohormone sensor.

13. The system of claim 11, wherein the neural activity sensor comprises an action potential recorder.

14. The system of claim 1, wherein the heart failure detector comprises a heart rate variability detector.

15. The system of claim 1, wherein the heart failure detector comprises a renal function sensor.

16. The system of claim 15, wherein the renal function sensor includes a renal output sensor.

17. The system of claim 15, wherein the renal function sensor includes a filtration rate sensor.

18. The system of claim 15, wherein the renal function sensor includes a chemical sensor adapted to sense angiotensin II levels.

19. The system of claim 1, wherein the heart failure detector comprises an acoustic sensor.

20. The system of claim 19, wherein the acoustic sensor comprises an accelerometer.

21. The system of claim 19, wherein the acoustic sensor comprises a microphone.

22. The system of claim 19, wherein the acoustic sensor comprises a heart sound sensor.

23. The system of claim 19, wherein the acoustic sensor comprises a respiratory sound sensor.

24. The system of claim 1, further comprising an external device communicatively coupled to the implantable CRM device, the external device including an external user input receiving the external user command.

25. The system of claim 24, further comprising:
a remote device receiving signals including at least one of the alert signal and the external user command; and
a network coupled between the external device and the remote device to provide for bidirectional communication between the external device and the remote device.

26. The system of claim 25, wherein the remote device comprises an emergency response module adapted to contact an emergency response unit in response to the at least one of the alert signal and the external user command.

27. The system of claim 25, wherein the remote device comprises a remote signal processor to process the received signals using at least one predetermined algorithm.

28. The system of claim 25, wherein the remote device further comprises a remote user interface providing for monitoring of the processed received signals and entry of remote user commands.

29. The system of claim 25, wherein the remote device further comprises a remote device controller generating commands controlling one or more of the transdermal drug delivery device, the implantable CRM device, and the external device based on the received signals and the remote user commands.

30. The system of claim 29, wherein the heart failure detector detects a signal indicative of the acute decompensated heart failure, the received signals comprise the signal indicative of the acute decompensated heart failure, and the remote device controller is adapted to generate the commands controlling the one or more of the transdermal drug delivery device, the implantable CRM device, and the external device further based on the signal indicative of the acute decompensated heart failure.

31. The system of claim 1, wherein the implantable CRM device further comprises a drug level detector, coupled to the drug delivery controller, to produce an indication of a blood drug concentration, and wherein the drug delivery controller controls the transdermal drug delivery device further based on the indication of the blood drug concentration.

32. The system of claim 31, wherein the drug level indicator comprises a blood drug level detector to measure the blood drug concentration.

33. The system of claim 31, wherein the drug level indicator comprises a respiratory sensor to sense a biological signal as the indication of the blood drug concentration.

34. The system of claim 1, wherein the transdermal drug delivery device comprises a drug reservoir containing a drug including one or more pharmaceutical agents.

35. The system of claim 34, wherein transdermal drug delivery device further comprises at least one skin contact electrode for transdermal drug delivery.

36. The system of claim 35, wherein the one or more pharmaceutical agents comprises one or more pharmaceutical agents for treating one or more symptoms related to the acute decompensated heart failure.

37. The system of claim 34, wherein the transdermal drug delivery device further comprises a reservoir drug level detector to detect a level of the drug contained in the drug reservoir and produce a warning signal if the level of the drug contained in the drug reservoir reaches a predetermined minimum level.

38. A method, comprising:
detecting an acute decompensated heart failure using an implantable cardiac rhythm management (CRM) device;
detecting an external user command controlling a drug delivery, the external user command transmitted to the implantable CRM device from an external device;
producing a drug control signal based on at least the acute decompensated heart failure and the external user command;
transmitting the drug control signal to an transdermal drug delivery device; and
delivering a drug from the transdermal drug delivery device.

39. The method of claim 38, further comprising producing an alert signal in response to a detection of the acute decompensated heart failure, and wherein producing the drug control signal comprises producing the drug control signal based on at least the alert signal and the external user command.

40. The method of claim 39, wherein producing the alert signal comprises producing a signal indicative of a degree of the acute decompensated heart failure.

41. The method of claim 38, further comprising:
acquiring information regarding symptoms of the acute decompensated heart failure; and
issuing the external user command based on the information.

42. The method of claim 38, wherein detecting the acute decompensated heart failure comprises:
detecting an impedance; and
indicating the acute decompensated heart failure when the impedance is outside of a predetermined impedance range.

43. The method of claim 42, wherein detecting the impedance includes detecting an impedance indicative of minute ventilation.

44. The method of claim 42, wherein detecting the impedance includes detecting an impedance indicative of pulmonary edema.

45. The method of claim 38, wherein detecting the acute decompensated heart failure comprises:
detecting a pressure; and
indicating the acute decompensated heart failure when the pressure is outside a predetermined pressure range.

46. The method of claim 45, wherein detecting the pressure comprises detecting left atrial pressure, and indicating the acute decompensated heart failure comprises indicating the acute decompensated heart failure when the left atrial pressure exceeds a predetermined threshold level.

47. The method of claim 45, wherein detecting the pressure comprises detecting left ventricular pressure, and indicating the acute decompensated heart failure comprises indicating the acute decompensated heart failure when the left ventricular pressure falls below a predetermined threshold level.

48. The method of claim 45, wherein detecting the pressure comprises detecting as arterial pressure, and indicating the acute decompensated heart failure comprises indicating the acute decompensated heart failure when arterial pressure falls below a predetermined threshold level.

49. The method of claim 45, wherein detecting the pressure comprises detecting a pulmonary pressure, and indicating the acute decompensated heart failure comprises indicating the acute decompensated heart failure when the pulmonary pressure exceeds a predetermined threshold level.

50. The method of claim 38, wherein detecting the acute decompensated heart failure comprises:
detecting a signal indicative of stroke volume; and
indicating the acute decompensated heart failure when the signal indicative of stroke volume falls below a predetermined threshold level.

51. The method of claim 38, wherein detecting the acute decompensated heart failure comprises:
detecting activities of at least one of a sympathetic nerve and a parasympathetic nerve; and
indicating the acute decompensated heart failure when the activity level of the sympathetic nerve exceeds a predetermined threshold level.

52. The method of claim 38, wherein detecting the acute decompensated heart failure comprises:
detecting a heart rate variability; and
indicating the acute decompensated heart failure when the heart rate variability falls below a predetermined threshold level.

53. The method of claim 38, wherein detecting the acute decompensated heart failure comprises detecting renal function.

54. The method of claim 38, wherein detecting the acute decompensated heart failure comprises:
detecting heart sounds; and
indicating the acute decompensated heart failure when a parameter related to at least one of the detected heart sounds is out of a predetermined range.

55. The method of claim 54, wherein detecting the heart sounds comprises detecting third heart sounds (S3), and indicating the acute decompensated heart failure comprises indicating the acute decompensated heart failure when an S3 amplitude exceeds a predetermined threshold level.

56. The method of claim 38, wherein detecting the acute decompensated heart failure comprises:
detecting respiratory sounds; and
indicating the acute decompensated heart failure when a parameter related to at least one of the respiratory sounds is out of a predetermined range.

57. The method of claim 38, further comprising receiving the external user command by the external device.

58. The method of claim 57, further comprising:
acquiring information regarding symptoms of the acute decompensated heart failure; and
entering the external user command based on the information.

59. The method of claim 57, further comprising transmitting the external user command from the external device to a remote device though a network connecting the external device and the remote device.

60. The method of claim 59, further comprising notifying an emergency response unit upon reception of the external user command by the remote device.

61. The method of claim 59, further comprising:
notifying a remote user;
receiving a remote user command directing the drug delivery at the remote device;
transmitting the remote user command from the remote device to the external device through the network;
transmitting the remote user command from the external device to the implantable CRM device; and
detecting the remote user command transmitted from the external device to the implantable CRM device.

62. The method of claim 61, wherein producing the drug control signal comprises producing a drug control signal upon the detection of at least one of the acute decompensated heart failure, the external user command, and the remote user command.

63. The method of claim 62, further comprising:
acquiring information regarding symptoms of the acute decompensated heart failure; and
entering the remote user command based on the information.

64. The method of claim 38, wherein transmitting the drug control signal to the transdermal drug delivery device comprises transmitting through a telemetry link between the implantable CRM device and the transdermal drug delivery device.

65. The method of claim 38, wherein transmitting the drug control signal to the transdermal drug delivery device comprises transmitting a voltage signal representing the drug control signal via tissue conduction.

66. The method of claim 38, further comprising verifying whether a sufficient amount of the drug has been delivered.

67. The method of claim 66, wherein verifying whether the sufficient amount of the drug has been delivered comprises detecting a signal indicative of a concentration of the drug in blood.

68. The method of claim 67, wherein detecting the signal indicative of a concentration of the drug in blood comprises at least one of:
measuring blood drug concentration;
sensing a respiratory signal; and
measuring heart rate.

69. The method of claim 68, further comprises;
producing an insufficiency alert signal if the concentration of the of the drug in blood is below a predetermined level;
transmitting the insufficiency alert signal from the transdermal drug delivery device to the implantable CRM device; and
detecting the insufficiency alert signal transmitted from the transdermal drug delivery device to the implantable CRM device, wherein producing the drug control signal comprises producing the drug control signal upon the detection at least one of the acute decompensated heart failure, the external user command, and the insufficiency alert signal.

70. The method of claim 38, wherein delivery the drug comprises delivering one or more pharmaceutical agents for treating one or more symptoms related to the acute decompensated heart failure.

71. The method of claim 70, further comprising:
checking a level of the drug contained in the transdermal drug delivery device; and
producing a warning signal if the level of the drug reaches a predetermined minimum level.
